(19) United States
(12) Patent Application Publication
(10) Pub. No.: US 2026/0271608 A1
(43) Pub. Date: Sep. 10, 2026

(54) METHOD OF PRODUCING PHOTOELECTRIC CONVERSION ELEMENT

(71) Applicant: CANON KABUSHIKI KAISHA, Tokyo (JP)

(72) Inventors: NOBUHIRO NAKAMURA, Shizuoka (JP); TSUTOMU NISHIDA, Shizuoka (JP); MIZUKI SATO, Chiba (JP); TATSUYA OHSAWA, Shizuoka (JP)

(21) Appl. No.: 19/657,808

(22) Filed: Apr. 24, 2026

Related U.S. Application Data
(63) Continuation of application No. PCT/JP2024/038136, filed on Oct. 25, 2024.

(30) Foreign Application Priority Data
Oct. 27, 2023 (JP) 2023-184750
Oct. 27, 2023 (JP) 2023-184756
Oct. 27, 2023 (JP) 2023-184761
Dec. 21, 2023 (JP) 2023-216294
Dec. 21, 2023 (JP) 2023-216296
Dec. 21, 2023 (JP) 2023-216299
Feb. 16, 2024 (JP) 2024-022244
Feb. 16, 2024 (JP) 2024-022246
Feb. 16, 2024 (JP) 2024-022251
May 21, 2024 (JP) 2024-082826
Oct. 23, 2024 (JP) 2024-186504

(51) Int. Cl. H10K 71/13; H10K 30/40; H10K 30/57; H10K 30/86; H10K 85/30; H10K 85/50; H10K 85/60

(52) U.S. Cl. CPC H10K 71/13; H10K 30/40; H10K 30/86; H10K 85/326; H10K 85/331; H10K 85/371; H10K 85/50; H10K 85/633; H10K 30/57

(57) ABSTRACT

A method of producing a photoelectric conversion element improved in photoelectric conversion efficiency. The photoelectric conversion element includes a first electrode, a photoelectric conversion layer containing a crystal having a perovskite structure, a charge-transporting layer, and a second electrode. The method includes applying a charge-transporting layer coating liquid onto a surface of the photoelectric conversion layer to form the charge-transporting layer. The charge-transporting layer coating liquid contains an alcohol liquid, a resin, and a charge-transporting particle.

### CROSS-REFERENCE TO RELATED APPLICATIONS

This application is a Continuation of International Patent Application No. PCT/JP2024/038136, filed Oct. 25, 2024, which claims the benefit of Japanese Patent Application No. 2023-184761, filed Oct. 27, 2023, Japanese Patent Application No. 2023-184756, filed Oct. 27, 2023, Japanese Patent Application No. 2023-184750, filed Oct. 27, 2023, Japanese Patent Application No. 2023-216294, filed Dec. 21, 2023, Japanese Patent Application No. 2023-216296, filed Dec. 21, 2023, Japanese Patent Application No. 2023-216299, filed Dec. 21, 2023, Japanese Patent Application No. 2024-022244, filed Feb. 16, 2024, Japanese Patent Application No. 2024-022251, filed Feb. 16, 2024, Japanese Patent Application No. 2024-022246, filed Feb. 16, 2024, Japanese Patent Application No. 2024-082826, filed May 21, 2024, and Japanese Patent Application No. 2024-186504, filed Oct. 23, 2024, all of which are hereby incorporated by reference herein in their entirety.

### BACKGROUND

### Field of the Technology

The present disclosure relates to a method of producing a photoelectric conversion element.

### Description of the Related Art

In order to solve a disadvantage of the depletion of fossil energy and a global environmental disadvantage caused by the use of the fossil energy, investigations on a renewable and clean alternative energy source, such as solar energy, wind power, or water power, have been actively performed. In particular, an interest in a solar cell that directly changes sunlight into electrical energy has been increasing. The term “solar cell” as used herein means a battery that generates a current-voltage through utilization of a photovoltaic effect in which light energy is absorbed from sunlight to generate an electron and a hole.

Currently, an n-p diode-type silicon (Si) single crystal-based solar cell having a light energy conversion efficiency of more than 20% is widely known, and is actually used in solar power generation. However, the solar cell requires a high temperature treatment step and the price of a material itself is high, and hence there is a disadvantage in that the cost per unit electric power is high. In addition, there is a disadvantage with its supply property in terms of a silicon resource.

Meanwhile, a solar cell using an organic material (hereinafter also referred to as “organic solar cell”) does not require the high temperature treatment step, and can be produced in a so-called roll-to-roll system using a sheet-shaped substrate, and hence a cost reduction can be expected. However, further improvements in power generation efficiency and durability have been desired for practical use of the organic solar cell. In particular, the development of a perovskite solar cell including a crystal having a perovskite structure as a photoelectric conversion layer toward the practical use of the solar cell has been advanced because the cell is excellent in photoelectric conversion characteristic.

For example, in Japanese Patent Laid-Open No. 2018-170382, there is a description of a technology including incorporating an organic semiconductor and a polymer compound having a glass transition temperature of 100° C. or more into a hole-transporting layer to reduce its peeling from an electrode. In Japanese Patent Laid-Open No. 2022-168820, there is a description of a technology including forming, between a hole-transporting layer and a perovskite, a layer containing a phthalocyanine compound to improve conversion efficiency. In Q. Hu, et al., Sol. RRL, 2019, 3, 1800264, there is a description of a technology including mixing copper phthalocyanine and a conductive polymer into a hole-transporting layer to improve conversion efficiency.

According to investigations made by the inventors, it has been found that there is room for improvement in photoelectric conversion efficiency in each of the photoelectric conversion elements described in Japanese Patent Laid-Open No. 2018-170382, Japanese Patent Laid-Open No. 2022-168820, and Q. Hu, et al., Sol. RRL, 2019, 3, 1800264.

### SUMMARY

The present disclosure is directed to providing a method of producing a photoelectric conversion element improved in photoelectric conversion efficiency.

The above-mentioned provision of the method is achieved by the present disclosure described below. That is, the present disclosure is directed to a method of producing a photoelectric conversion element comprising a first electrode, a photoelectric conversion layer containing a crystal having a perovskite structure, a charge-transporting layer, and a second electrode, the method comprising applying a charge-transporting layer coating liquid onto a surface of the photoelectric conversion layer to form the charge-transporting layer, wherein the charge-transporting layer coating liquid contains an alcohol liquid, a resin, and a charge-transporting particle.

Features of the present disclosure will become apparent from the following description of embodiments with reference to the attached drawings.

### BRIEF DESCRIPTION OF THE DRAWINGS

FIG. 1 is a schematic sectional view in the thickness direction of a photoelectric conversion element produced by a method of producing a photoelectric conversion element of the present disclosure.

FIG. 2 is a perspective view for schematically illustrating an example of a moving body including the photoelectric conversion element produced by the method of producing a photoelectric conversion element of the present disclosure.

FIG. 3 is a perspective view for schematically illustrating an example of a building material including the photoelectric conversion element produced by the method of producing a photoelectric conversion element of the present disclosure.

### DESCRIPTION OF THE EMBODIMENTS

### One Embodiment

One embodiment is directed to a method of producing a photoelectric conversion element.

The method of producing a photoelectric conversion element of the present disclosure is a method of producing a photoelectric conversion element comprising a first electrode, a photoelectric conversion layer containing a crystal having a perovskite structure, a charge-transporting layer, and a second electrode, the method comprising applying a charge-transporting layer coating liquid onto a surface of the photoelectric conversion layer to form the charge-transporting layer, wherein the charge-transporting layer coating liquid contains an alcohol liquid, a resin, and a charge-transporting particle.

As a result of investigations, the inventors have found that a photoelectric conversion element having excellent photoelectric conversion efficiency can be produced by the above-mentioned production method. A photoelectric conversion element was produced by applying a coating liquid in which a charge-transporting particle was dispersed in a non-alcohol solvent onto the surface of a photoelectric conversion layer containing a crystal having a perovskite structure. When a cross section of the photoelectric conversion element was observed with an electron microscope under magnification, a void or a portion where the photoelectric conversion layer was not able to be covered with the charge-transporting particle was found between the photoelectric conversion layer and a layer containing the charge-transporting particle.

In contrast, it has been found that, in the case of the photoelectric conversion element formed by the production method of the present disclosure, the void or the portion where the photoelectric conversion layer cannot be covered with the charge-transporting particle is reduced, and hence a layer containing a charge-transporting particle that is denser and brought into close contact with the photoelectric conversion layer can be formed. Probably as a result of the foregoing, the movement of charge from the photoelectric conversion layer to the electrode is smoothened, and hence the photoelectric conversion efficiency is improved. The inventors have presumed the reason why the charge-transporting particle becomes a high-coverage layer, which is dense and brought into close contact with the photoelectric conversion layer, in the present disclosure to be as described below.

The charge-transporting layer coating liquid of the present disclosure is a liquid containing the alcohol liquid, the resin, and the charge-transporting particle. The charge-transporting particle is in a state of being dispersed in the alcohol liquid. For the charge-transporting layer, the charge-transporting layer coating liquid is applied onto the surface of the photoelectric conversion layer, the alcohol liquid volatilizes, and a layer containing the charge-transporting particle and the resin is formed.

In the applied charge-transporting layer coating liquid, as the alcohol liquid volatilizes, a distance between the charge-transporting particles shortens. At this time, the resin intervenes between the charge-transporting particles, and hence the particle dispersion state is stabilized by the steric barrier of the resin. Thus, excessive aggregation in the process of the shortening of the inter-particle distance is prevented, and hence the particle is stably deposited on the photoelectric conversion layer to form a high-coverage layer. In addition, the alcohol liquid has both of a hydrophilic moiety formed of a hydroxy group and a hydrophobic moiety formed of a hydrocarbon in a well-balanced manner, and hence the alcohol liquid stably intervenes between the charge-transporting particles and contributes to the prevention of excessive aggregation of the particles during coating film formation. Further, since the crystal having a perovskite structure of the photoelectric conversion layer has a hydrogen-bonding surface, it has good compatibility with the alcohol liquid by virtue of its hydrogen-bonding interaction therewith.

In the case of a non-alcohol, its compatibility with the crystal having a perovskite structure is poor. Accordingly, the molecules of the non-alcohol try to aggregate with each other, and as a result, the aggregation of the charge transporting particles and the generation of a portion where the photoelectric conversion layer is not covered with the charge-transporting particle are promoted. The alcohol liquid has satisfactory compatibility with each of the crystal having a perovskite structure and the charge-transporting particle, and hence excessive aggregation of the particles is not caused in the coating film formation process in which the alcohol liquid volatilizes. When the respective configurations synergistically affect each other as in the above-mentioned mechanism, the charge-transporting particle may become a high-coverage layer, which is dense and brought into close contact with the photoelectric conversion layer, to improve the photoelectric conversion efficiency.

The effects of the present disclosure can be achieved when the respective configurations synergistically affect each other as in the above-mentioned mechanism.

The present disclosure is described in detail below by way of illustrative embodiments. The present disclosure is not limited to the following embodiments, and the following embodiments, which are appropriately changed, modified, and the like based on the ordinary knowledge of a person skilled in the art without departing from the gist of the present disclosure, are also encompassed within the scope of the present disclosure.

The term “layer” as used herein means not only a layer having a clear boundary or a layer having a flat thin film shape but also a layer having a concentration gradient in which the concentration of a constituent element gradually changes, or a layer that may form a complicatedly intricate structure together with another layer. In addition, the elemental analysis of the layer may be performed by, for example, performing the TOF-SIMS/FE-TEM/EDS line analysis measurement of a cross section of the photoelectric conversion element and observing the element distribution of a specific element. The analysis of each layer may be performed by peeling and removing a layer from a completed photoelectric conversion element to expose the layer to be analyzed. In addition, the analysis of a compound in the charge-transporting layer coating liquid may be performed by performing mass and structural analyses through ¹H-NMR, GPC, MALDI-TOF-MS, IR, gas chromatography, liquid chromatography, or elemental analysis, such as XPS or EDX.

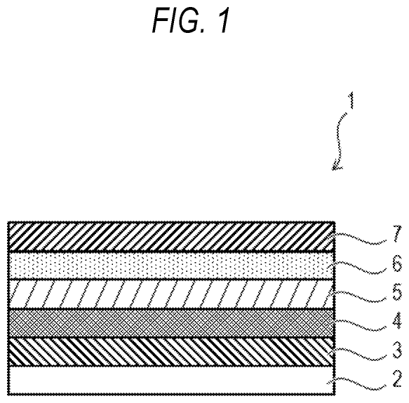

The photoelectric conversion element produced by the method of producing a photoelectric conversion element of the present disclosure includes the first electrode, the photoelectric conversion layer containing the crystal having a perovskite structure, the charge-transporting layer, and the second electrode. FIG. 1 is a sectional view for schematically illustrating the configuration of a photoelectric conversion element 1 produced by the method of producing a photoelectric conversion element of the present disclosure. The photoelectric conversion element 1 includes, on a substrate 2, a second electrode 3, an electron-transporting layer 4, a photoelectric conversion layer 5, a charge-transporting layer 6, and a first electrode 7. One of the first electrode 7 and the second electrode 3 is an anode, and the other is a cathode. A current can be extracted by connecting the first electrode 7 and the second electrode 3 with an external circuit.

The photoelectric conversion layer 5 is excited by light that has entered the layer through the substrate 2, the second electrode 3, and the electron-transporting layer 4, or the first electrode 7 and the charge-transporting layer 6 to generate an electron or a hole. That is, the photoelectric conversion layer 5 generates a current between the first electrode 7 and the second electrode 3. The electron-transporting layer 4 is a layer arranged between the photoelectric conversion layer 5, and the two electrodes (the second electrode 3 and the first electrode 7), and may be omitted in some cases. A form in which the plurality of electron-transporting layers 4 and photoelectric conversion layers 5 are laminated may be adopted. Such form may also be referred to as “tandem structure.”

The photoelectric conversion element of the present disclosure is characterized by including: the first electrode 7; the second electrode 3; the photoelectric conversion layer 5 arranged between the first electrode 7 and the second electrode 3, the photoelectric conversion layer containing a crystal having a perovskite structure; and the charge-transporting layer 6 between the photoelectric conversion layer 5 and the first electrode 7. In addition, in order to improve the photoelectric conversion efficiency, a tandem type in which the photoelectric conversion elements 1 are laminated may be adopted. The kind of the photoelectric conversion element to be laminated is not limited, and for example, a silicon solar cell or a CIGS solar cell may be adopted in addition to a perovskite solar cell using a crystal having a perovskite structure in the photoelectric conversion layer 5.

A method of forming each of the layers including the photoelectric conversion layer and charge-transporting layer of the photoelectric conversion element 1 is, for example, a coating method or a vapor deposition method. Examples of the coating method include dip coating, spin coating, spray coating, ink jet coating, meniscus coating, screen coating, roll coating, die coating, blade coating, curtain coating, and wire bar coating. The coating method is a method including preparing a coating liquid for each layer to be described later, applying the liquid in the desired order of layers, and drying the liquid. A desired method may be selected as such forming method in accordance with each layer.

The respective steps are described below.

### [Step of Forming Charge-Transporting Layer]

The method of producing a photoelectric conversion element of the present disclosure includes the step of applying the charge-transporting layer coating liquid onto the surface of the photoelectric conversion layer 5 to form the charge-transporting layer 6, and the charge-transporting layer coating liquid contains the alcohol liquid, the resin, and the charge-transporting particle.

The thickness of the charge-transporting layer 6 is preferably 1 to 1,000 nm, more preferably 5 to 500 nm, particularly preferably 10 to 200 nm.

In the method of producing a photoelectric conversion element of the present disclosure, the charge-transporting layer coating liquid contains the alcohol liquid, the resin, and the charge-transporting particle (serving as a charge-transporting material). In the charge-transporting layer coating liquid of the present disclosure, the charge-transporting material (charge-transporting particle) is dispersed in the coating liquid to be present in a particulate form. When the charge-transporting layer is formed in a particulate form, high crystallinity is maintained in the charge-transporting layer, and hence a high charge-transporting ability inherent therein can be expressed.

A particle shape having a small aspect ratio, such as a substantially spherical shape, a rugby-ball shape, a cylindrical shape, or a flat shape, is preferred as the particle shape of the charge-transporting particle. For example, when particles each having a large aspect ratio such as needle-shaped particles are adopted, the particles need to be deposited in a unified orientation to improve coverage.

That is, even when the charge-transporting particles can be deposited on the photoelectric conversion layer in a stable dispersed state without causing excessive aggregation, a portion where the photoelectric conversion layer cannot be covered with the charge-transporting particles is liable to remain owing to their shapes. In addition, the particle is liable to stack on any other particle, and hence a void is liable to remain between the photoelectric conversion layer and a layer containing the charge-transporting particle. Thus, the effects of the present application become smaller.

The aspect ratio of the charge-transporting particle is preferably 5 or less, more preferably 3 or less. A particle shape that reduces the degrees of the effects of the present application is, for example, a needle shape or a fibrous shape. The aspect ratio is larger than 10.

Specific examples of the charge-transporting particle include a phthalocyanine pigment, an azo pigment, a lake pigment, a quinacridone pigment, a dioxazine pigment, a perylene pigment, and an isoindolinone pigment.

In the method of producing a photoelectric conversion element of the present disclosure, the charge-transporting particle is preferably a particle containing a cyclic conjugated compound in which a plurality of pyrrole rings are bonded by conjugated bonds. The cyclic conjugated compound in which a plurality of pyrrole rings are bonded by conjugated bonds has a planar structure, and the particle is formed of an aggregate of crystallites in which the planar structures overlap each other. The crystallites each have a large-polarity surface containing a nitrogen atom, where a π-electron cloud is present, on a surface corresponding to the vertical direction of the planar structure, and a non-polar surface on a surface corresponding to the lateral direction of the planar structure.

Accordingly, a surface rich in polarity and a non-polar surface are present on the particle formed by the aggregation of the crystallites. An alcohol has both of a hydrophilic moiety and a hydrophobic moiety, and hence the particle containing the cyclic conjugated compound in which a plurality of pyrrole rings are bonded by conjugated bonds is suitably dispersed by the alcohol, and the coverage of the charge-transporting layer on the photoelectric conversion layer is improved. In addition, the particle containing the cyclic conjugated compound in which a plurality of pyrrole rings are bonded by conjugated bonds is preferred because the particle exhibits a high charge-transporting ability.

In the method of producing a photoelectric conversion element of the present disclosure, the charge-transporting particle is more preferably a particle containing a phthalocyanine compound. Further, the charge-transporting particle is still more preferably a particle having a structure represented by the following formula (Pc-2). With such configuration, the charge-transporting particle easily interacts with an alcohol and a resin, and the coverage is easily improved. In addition, the above-mentioned configuration is more preferred because its crystallinity is satisfactory and its charge-transporting property is high.

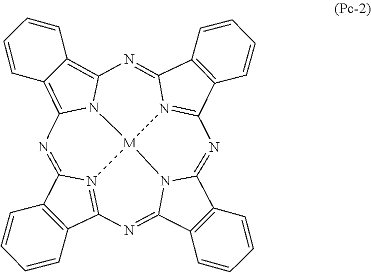

In the formula (Pc-2), M represents H₂, a metal atom having a ligand, or a metal atom free of a ligand. In the present disclosure, the structure of a chemical substance may be determined by nuclear magnetic resonance (NMR).

In particular, when M in the formula (Pc-2) represents H₂, the formula (Pc-2) is represented by the following formula (Pc-1).

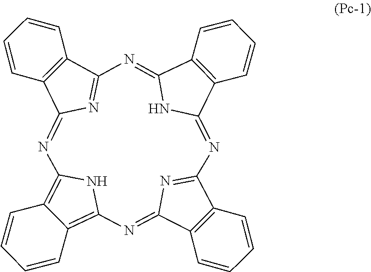 [SMILES: c1ccc2c(c1)-c1nc-2nc2[nH]c(nc3nc(nc4[nH]c(n1)c1ccccc41)-c1ccccc1-3)c1ccccc21]

Specific examples of the resin include a polyacetal resin, an acrylic resin, a polyarylate resin, a polycarbonate resin, a polyvinyl acetate resin, a polyester resin, a polyamide resin, a polyurethane resin, and a polystyrene resin.

In the method of producing a photoelectric conversion element of the present disclosure, the resin is preferably a resin having a Lewis basic functional group. The Lewis basic functional group acts electronically on the charge-transporting particle, and hence an interaction between the particle and the resin is enhanced. Thus, the resin easily intervenes between the particles, and hence the effect of steric barrier stabilization by the resin is easily obtained. A case in which the particle is a cyclic conjugated compound in which a plurality of pyrrole rings are bonded by conjugated bonds is more effective because the particle easily interacts electronically with the Lewis basic functional group.

Specific examples of the Lewis basic functional group include a hydroxy group, a halogen group, a sulfo group, an amino group, a carbonyl group, an ester group, an ether group, a carboxyl group, an aldehyde group, a methoxy group, an amide group, a sulfide group, a cyano group, a thienyl group, a thienylidene group, a pyridyl group, a furyl group, a pyrazolyl group, an imidazolyl group, an oxazolyl group, and a thiazolyl group. Of those, a hydroxy group, a carbonyl group, an ether group, an ester group, a pyridyl group, and a thienylidene group are preferred from the viewpoint of an electronic interaction.

In the method of producing a photoelectric conversion element of the present disclosure, the resin preferably has at least one functional group selected from the group consisting of a hydroxy group, a carbonyl group, an ether group, an ester group, a pyridyl group and a thienylidene group. In the present disclosure, the functional group of the resin may be identified by, for example, nuclear magnetic resonance (NMR). The thienylidene group has a structure represented by the following formula (A).

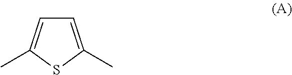

In particular, the resin more preferably has at least two of those functional groups from the viewpoint of the ease of an interaction. One kind of resin may have different functional groups, or two kinds of resins having different functional groups may be mixed. The Lewis basic functional group is preferably included in the repeating structure of the resin.

Specific examples of the resin having a Lewis basic functional group to be preferably used in the present disclosure include the following resins: polyvinyl butyral, poly(4-vinylpyridine), poly(vinyl chloride), poly(vinylidene fluoride), polyacrylonitrile, poly(vinylidene fluoride-co-hexafluoropropylene), poly(acrylonitrile-co-butadiene), poly(styrene-co-acrylonitrile), polychloroprene, poly(4-chlorostyrene), poly(methyl methacrylate), polyvinyl acetate, polyethyleneimine, polyvinyl alcohol, polyacrylic acid, poly(sodium 4-styrenesulfonate), poly(allylamine hydrochloride), sodium polyacrylate, poly(4-styrenesulfonic acid), poly(N-isopropylacrylamide), poly(2-ethyl-2-oxazoline), poly(ethylene-alt-maleic anhydride), poly(2-acrylamido-2-methyl-1-propanesulfonic acid), poly(vinyl sulfate) potassium salt, polyanetholesulfonic acid sodium salt, poly(2-(dimethylamino)ethyl methacrylate) methyl chloride quaternary salt, poly(methyl vinyl ether), poly(2-propylacrylic acid), polyvinylpyrrolidone, polypropylene glycol, poly(propylene carbonate), polyvinyl acetate, poly(tetrahydrofuran), nylon-6, poly(ethylene-co-vinyl acetate), poly(propylene glycol) bis(2-aminopropyl ether), poly(bisphenol A carbonate), poly(1,4-butylene adipate), poly(4-vinylphenol), poly(propylene glycol) monobutyl ether, poly(glycidyl methacrylate), poly(butyl acrylate), poly(ethylene succinate), poly(propylene glycol) methacrylate, nylon 11, nylon 12, poly(2-ethylhexyl acrylate), poly(bisphenol A carbonate), poly(propylene glycol) bis(2-aminopropyl ether), polyetherimide, poly(vinyl formal), poly(vinyl methyl ketone), poly(3-hexylthiophene-2,5-diyl), polyaniline, and poly(3,4-ethylenedioxythiophene) and poly(styrenesulfonate) composite (PEDOT:PSS). Of those, polyvinyl butyral, polymethyl methacrylate, poly(3-hexylthiophene-2,5-diyl), polyvinyl acetate, polyvinyl alcohol, polyacrylic acid, poly(2-propylacrylic acid), poly(butyl acrylate), and poly(4-vinylpyridine) are more preferred, and a polyvinyl acetal resin and a polyvinyl butyral resin are particularly preferred from the viewpoint of an electronic interaction. In the method of producing a photoelectric conversion element of the present disclosure, the resin preferably includes a polyvinyl acetal resin, and more preferably includes a polyvinyl butyral resin. Any such resin easily interacts with the charge-transporting particle.

In the method of producing a photoelectric conversion element of the present disclosure, the glass transition temperature of the resin is preferably 95° C. or less. When the glass transition temperature falls within the range, the resin is easily brought into close contact with the charge-transporting particle, and an improvement in coverage by an interaction therebetween becomes more effective. In the present disclosure, the glass transition temperature may be determined with a differential scanning calorimeter (DSC).

The molecular weight of the resin preferably falls within the range of 1,000 to 1,000,000 in terms of weight-average molecular weight from the viewpoints of dispersibility and film formability.

In the method of producing a photoelectric conversion element of the present disclosure, the mass of the charge-transporting particle in the charge-transporting layer coating liquid is preferably 5 to 20 times with respect to the mass of the resin in the charge-transporting layer coating liquid. With such configuration, the resin easily interacts with the charge-transporting particle, and the coverage is easily improved. When the mass is more than 20 times, an improvement in coverage by the interaction of the resin tends to be insufficient. When the mass is less than 5 times, charge transfer from the photoelectric conversion layer to the particle becomes insufficient, and hence it becomes difficult to improve the photoelectric conversion efficiency of the photoelectric conversion element. In the present disclosure, the ratio between the mass of the charge-transporting particle and the mass of the resin may be identified, for example, by taking a small amount of the charge-transporting layer coating liquid, dissolving the charge-transporting layer coating liquid in deuterated DMSO at a low concentration, and determining a peak ratio derived from each structure by NMR measurement.

The charge-transporting layer coating liquid contains the alcohol liquid. The alcohol liquid has a function as a dispersion medium for the charge-transporting particle in the coating liquid. In addition, the alcohol liquid has a low surface tension and can exhibit satisfactory wettability of the charge-transporting layer coating liquid on the surface of the photoelectric conversion layer containing the crystal having a perovskite structure.

In the method of producing a photoelectric conversion element of the present disclosure, the alcohol liquid preferably contains an aliphatic alcohol, and particularly preferably contains an alcohol having 5 or less carbon atoms. When the number of carbon atoms falls within the range, balance between the hydrophobic moiety and hydrophilic moiety of the alcohol liquid is satisfactory, and hence the alcohol liquid can more suitably contribute to an improvement in coverage. The alcohol having 5 or less carbon atoms is preferred particularly because a particle containing a cyclic conjugated compound in which a plurality of pyrrole rings are bonded by conjugated bonds can be suitably dispersed in the charge-transporting layer coating liquid, and hence the coverage can be improved. In the present disclosure, a chemical substance such as an alcohol liquid may be identified by, for example, nuclear magnetic resonance (NMR).

Two or more kinds of alcohol liquids in the charge-transporting layer coating liquid may be mixed. In addition, the charge-transporting layer coating liquid may contain a solvent except the alcohol liquid. When the charge-transporting layer coating liquid contains a solvent except the alcohol liquid, the mass of the alcohol liquid with respect to the mass of all solvents in the charge-transporting layer coating liquid is preferably 40% or more because the above-mentioned effect is easily expressed.

In the method of producing a photoelectric conversion element of the present disclosure, the particle diameter of the charge-transporting particle in the charge-transporting layer coating liquid is preferably 1.0×10¹ to 5.0×10² nm. When the particle diameter falls within the above-mentioned range, an improvement in dispersion stability of the charge-transporting particle during coating becomes more effective, and hence the coverage is easily improved.

In the present disclosure, the particle diameter of the charge-transporting particle in the charge-transporting layer coating liquid may be measured with Zetasizer Nano ZS (manufactured by Malvern Panalytical Ltd.). The particle diameter may be measured with the apparatus by a dynamic light scattering method. First, a particle to be measured is diluted to prepare a dilution so that its solid-liquid ratio may become 0.10 mass % (±0.02 mass %). The dilution is collected in a quartz cell and the cell is loaded into a measurement section. The same liquid as a solvent for forming the charge-transporting layer coating liquid is prepared and used as a diluting liquid. For measurement conditions, the refractive index and viscosity of a solvent for dispersing the particle at 20° C. are input with control software Zetasizer software 6.30, and its particle diameter is measured under such a condition that the temperature of the liquid is 20° C., followed by the determination of its Z-average particle diameter. In the case of a mixed solvent, the mass-average value of dispersion media to be mixed is adopted.

The particle diameter of the charge-transporting particle may be changed by dispersing the charge-transporting layer coating liquid with a paint shaker, and the particle diameter may be reduced by increasing a dispersion time. In addition, the particle diameter may be reduced by further subjecting the charge-transporting layer coating liquid to a centrifuge.

The charge-transporting layer coating liquid more preferably contains an aromatic compound having a hydroxy group, which is different from the charge-transporting particle and the resin. When the charge-transporting layer coating liquid contains the aromatic compound having a hydroxy group, an improvement in dispersion stability of the charge-transporting particle during coating becomes more effective, and hence the coverage is easily improved.

The charge-transporting layer may be formed by preparing a charge-transporting layer coating liquid containing the above-mentioned respective materials and a solvent, forming a coating film of the coating liquid on the photoelectric conversion layer, and drying the coating film. A method of forming the coating film is, for example, dip coating, spin coating, spray coating, ink jet coating, meniscus coating, screen coating, roll coating, die coating, blade coating, curtain coating, or wire bar coating.

The photoelectric conversion element of the present disclosure may include a second charge-transporting layer between the first electrode and the charge-transporting layer. When the photoelectric conversion element includes the second charge-transporting layer, the transfer of carriers to an electrode may be facilitated.

### [Substrate]

The photoelectric conversion element 1 of the present disclosure may include the substrate 2, and examples thereof include a transparent glass substrate made of soda-lime glass or alkali-free glass, a ceramic substrate, and a transparent plastic substrate. When light is taken in from the first electrode 7 side, an opaque material may be used as the substrate 2, and when light is taken in from the second electrode 3 side, the substrate 2 is formed of a transparent material.

### [First Electrode and Second Electrode]

For example, a vacuum deposition method, a sputtering method, or any other known method may be used as a method of forming each of the first electrode and the second electrode. Examples thereof include, but not limited to, a sputtering method, a vacuum deposition method, a vapor phase growth method (CVD method), and a spray pyrolysis deposition method (SPD method).

A material for the first electrode 7 or the second electrode 3 is not particularly limited, and a material that has hitherto been known may be used. Examples thereof include: metals, such as gold, silver, titanium, and copper; sodium; a sodium-potassium alloy; lithium; magnesium; carbon; a carbon nanotube; aluminum; a magnesium-silver mixture; a magnesium-indium mixture; an aluminum-lithium alloy; an Al/Al₂O₃ mixture; and an Al/LiF mixture.

Examples of a transparent electrode material include conductive transparent materials, such as CuI, indium tin oxide (ITO), SnO₂, aluminum zinc oxide (AZO), indium zinc oxide (IZO), gallium zinc oxide (GZO), fluorine-doped tin oxide (FTO) and antimony-doped tin oxide (ATO), and conductive transparent polymers. Those materials may be used alone or in combination thereof.

At least one electrode of the first electrode 7 or the second electrode 3 on a light incident side is a transparent electrode, and the other may be a transparent electrode or may also serve as a reflective layer formed of a light reflective material, or may be a transparent electrode including a reflective layer on a side opposite to the light incident side. When the first electrode 7 is on the light incident side, the second electrode 3 and the substrate 2 may be a transparent electrode and a reflective layer, respectively. The electrode may be a patterned electrode. When one, or each of both, of the first electrode and the second electrode is a transparent electrode, the thickness of the transparent electrode is preferably 0.03 to 3 μm.

When a solar cell is produced, cutting processing is generally performed for circuit formation between steps. Examples of the cutting processing include mechanical patterning and laser patterning.

### <Modularization Step>

The element formed up to the electrode may be sealed. A method for the sealing is, for example, sealing with a resin or sealing with a film. Examples of a material used for the sealing include a silazane, a silicone rubber, a resin having a siloxane skeleton, and glass.

In addition, hairline treatment may be performed on the surface of the sealed element from the viewpoint of the suppression of adhesion between elements occurring during winding in a roll-to-roll system.

### [Photoelectric Conversion Layer]

A method of forming the photoelectric conversion layer includes a step of applying a liquid containing a material for the photoelectric conversion layer. Examples of an application method include a spin coating method, a blade coating method, a slit die coating method, a screen printing method, a bar coater method, a casting method, a printing transfer method, a dip-drawing method, an ink jet method, a spray method, and a vacuum vapor deposition method. The method is appropriately selected therefrom in accordance with properties of a photoelectric conversion layer to be produced, such as thickness control and orientation control.

Annealing treatment may be performed under reduced pressure or in an inert atmosphere (in a nitrogen or argon atmosphere) in order to remove a solvent or a dispersion medium from the applied liquid containing the material for the photoelectric conversion layer. The temperature of the annealing treatment is preferably 40 to 300° C., more preferably 50 to 150° C. The annealing treatment is preferably performed because materials for forming the respective layers may permeate each other at an interface between laminated layers to increase a contact area, and hence a short-circuit current can be increased in some cases.

The photoelectric conversion layer 5 contains the crystal having a perovskite structure. The crystal having a perovskite structure to be used in the present disclosure is preferably represented by the following general formula [1].

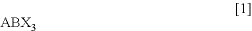

In the general formula [1], A represents a monovalent cation of an organic molecule or a metal atom, B represents a divalent metal cation, and X represents a monovalent halide anion.

In the case of the organic molecule, for example, A in the general formula [1] is preferably represented by CₚN_{q}Hᵣ (“p”, “q”, and “r” each represent a positive integer). Specific examples thereof include methylammonium and formamidinium.

In addition, the metal atom is not particularly limited, and lithium, cesium, sodium, potassium, and rubidium are preferred. Those organic molecules or metal atoms may be used alone or in combination thereof.

When the cation A to be included is too large to fit in a crystal having a three-dimensional perovskite structure, a crystal having a two-dimensional perovskite structure, a crystal having a 2.5-dimensional perovskite structure with properties of both the two-dimensional and three-dimensional perovskite structures, a two-layer crystal having three-dimensional and two-dimensional perovskite structures, or a crystal having a mixed three-dimensional/two-dimensional perovskite structure is formed, and any of the structures functions as the photoelectric conversion layer. The two-layer crystal having three-dimensional and two-dimensional perovskite structures refers to a crystal in which the crystals having three-dimensional and two-dimensional perovskite structures are layered as independent and separate layers. The crystal having a mixed three-dimensional/two-dimensional perovskite structure refers to a crystal having a structure in which both the regions or domains of crystals having two-dimensional or 2.5-dimensional layered and three-dimensional perovskite structures are mixed.

It is preferred that the crystal having a two-dimensional perovskite structure or the crystal having a 2.5-dimensional perovskite structure be represented by each of the following general formulae [2] to [4] (“n” represents a positive integer).

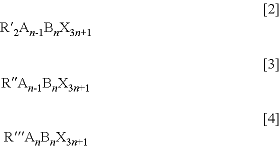

The general formula [2], the general formula [3], and the general formula [4] form perovskite structures of a Ruddlesden-Popper (RP) type, a Dion-Jacobson (DJ) type, and an Alternating cations in the interlayer (ACI) type, respectively.

R′, R″, and R″ in the general formulae [2] to [4] each are preferably represented by CₚNₘHₙ (“p”, “m”, and “n” each represent a positive integer), for example in the case of the organic molecule. In addition, A represents a cation of an organic molecule that may have a substituent or of a metal. Specifically, ethylammonium, propylammonium, n-butylammonium, n-hexylammonium, n-octylammonium, 1,6-hexanediammonium, iso-butylammonium, 3-(nonafluoro-tert-butyloxy) propylamine, 1,3-propanediammonium, 1,5-pentamethylenediamine, octyldiammonium, 2,2-(ethylenedioxy)bis(ethylammonium), 5-aminovaleric acid, 4-tert-butylammonium, N,N′-dimethylethylene-1,2-diammonium, 2,2,3,3,3-pentafluoropropylammonium, guanidinium, propylammonium, propargylamine, alkylammonium, cyclohexylmethylammonium, 4-(aminomethyl) piperidinium, piperidinium, pyrrolidinium, cyclohexylammonium, 4-fluorophenethylammonium, trifluoromethylbenzylammonium, pentafluorobenzylammonium, pentafluorophenylethylammonium, 4-methoxyphenethylammonium, imidazolium, pyridinium, 3-thiophenemethylammonium, 2-thiopheneethylammonium, 2-thiopheneformamidinium, 2-thiophenemethylammonium, 1-naphthylmethylammonium, 2-naphthylmethylammonium, phenethylammonium, phenylammonium, benzylammonium, 2,5-thiophenedimethylammonium, phenylpropylammonium, 1,4-phenylenedimethanamine, 3-phenyl-2-propen-1-ammonium, phenylbutylammonium, 4-tert-butylbenzylammonium, 3-(aminomethyl) piperidinium, and 4-(aminomethyl) piperidinium are preferred.

B in each of the general formulae [1] to [4] represents a metal atom, and examples thereof include lead, tin, bismuth, zinc, titanium, antimony, nickel, iron, cobalt, silver, copper, gallium, germanium, magnesium, calcium, indium, aluminum, manganese, chromium, molybdenum, and europium. Of those, lead, tin, and bismuth are preferred from the viewpoint of the overlap of electron orbits. Those metal atoms may be used alone or in combination thereof.

X in each of the general formulae [1] to [4] represents a halogen atom, and examples thereof include chlorine, bromine, and iodine. Those halogen atoms may be used alone or in combination thereof. Of those, a halogen atom is preferred because, when the halogen atom is incorporated into the perovskite structure, the above-mentioned crystal having a perovskite structure easily becomes soluble in an organic solvent, and hence the application to an inexpensive printing method or the like is enabled. Further, iodine is more preferred because the energy bandgap of the crystal having a perovskite structure narrows.

Specifically, as the crystal having a three-dimensional perovskite structure, the crystal having a two-dimensional perovskite structure, and the crystal having a mixed three-dimensional/two-dimensional perovskite structure, MAPbI₃, FAPbCl₃, FAPbI₃, MAPbIₓBr₃₋ₓ, MAPbIₓCl₃₋ₓ, Cs_{0.05}(MA_{0.17}FA_{0.83})_{0.95}Pb(I_{0.83}Br_{0.17})₃, {Csₓ₁(FAₓ₂MA₁₋ₓ₂)₁₋ₓ₁}ₓ₃Pb(Iₓ₄Br₁₋ₓ₄)ₓ₅, Cs_{0.05}FA_{0.88}MA_{0.07}PbI_{2.56}Br_{0.44}, (FAPbI₃)_{0.95}(MAPbBr₃)_{0.05}, (FAPbI₃)_{0.85}(MAPbBr₃)_{0.15}, CsPbI₃, CsPbBr₃, Csₓ(MA)₁₋ₓPbI₃, Csₓ(FA)₁₋ₓPbI₃, MAₓ(FA)₁₋ₓPbI₃, MA_{0.17}FA_{0.83}Pb(I_{0.83}Br_{0.17})₃, Cs_{0.15}FA_{0.85}PbI_{2.55}Br_{0.45}, (PEA)₂(MA)₂Pb₃I₁₀, (PTA)₂(MA)₄Pb₅I₁₆, (PEA)₂(MA)₄Pb₅I₁₆, (ThMA)₂(MA)₂Pb₃I₁₀, (3BBA)₂(MA)₂Pb₃I₁₀, (ThMA)₂(FA)₄Pb₅I₁₆, (4FPEA)₂(FA_{0.3}MA_{0.7})₄Pb₅I₁₆, (PDMA)FA₂Pb₃I₁₀, (3AMPY)(MA)₃Pb₄I₁₃, (PDMA)MA₅Pb₆I₁₉, (PDMA)MA₃Pb₄I₁₃, (TTDMA)MA₃Pb₄I₁₃, (TTDMA)MA₄Pb₅I₁₆, (BA_{0.9}PEA_{0.1})₂MA₄Pb₅I₁₆, (BA_{0.9}PEA_{0.1})₂MA₃Pb₄I₁₃, (4FPEA)₂MA₃Pb₄I₁₃, (4FPEA)₂MA₄Pb₅I₁₆, (BA)₂MA₂Pb₃I₁₀, (BA)₂MA₃Pb₄I₁₃, (TEA)₂MA₂Pb₃I₁₀, (BA)₂MA₄Pb₅I₁₆, (BA)₂MA₃Pb₄I₁₃, CsSnBr₃, CsSnI₃, FA_{0.75}MA_{0.25}Sn_{0.95}Ge_{0.05}I₃, FAMASnGeI₃, FASnBr₃, FASnI₃, MA₂Sn₃I₈, MASnBr₃, MASnGeI₃, and MASnI₃ are preferred. The A site, B site, or X site of each of the general formulae may be adjusted to be deficient or excessive in accordance with purposes, and the combinations of x1 to x5 may be changed in accordance with purposes. The combinations of x1 to x5 are, for example, as shown in Table 1. Particularly preferred ranges are 0.03≤x1≤0.10, 0.80≤x2≤0.96, 0.95≤x3≤1.05, 0.80≤x4≤0.96, and 2.95≤x5≤3.05. MACI may be included as a material for forming the crystal having a perovskite structure.

TABLE 1

| x1 | x2 | 1-x2 | x3 | x4 | 1-x4 | x5 |
|---|---|---|---|---|---|---|
| 0.05 | 0.83 | 0.17 | 1.00 | 0.83 | 0.17 | 3.00 |
| 0.05 | 0.83 | 0.17 | 0.99 | 0.83 | 0.17 | 2.99 |
| 0.05 | 0.83 | 0.17 | 0.98 | 0.83 | 0.17 | 2.98 |
| 0.05 | 0.83 | 0.17 | 0.97 | 0.83 | 0.17 | 2.97 |
| 0.05 | 0.83 | 0.17 | 0.96 | 0.83 | 0.17 | 2.96 |
| 0.05 | 0.83 | 0.17 | 1.01 | 0.83 | 0.17 | 3.01 |
| 0.05 | 0.83 | 0.17 | 1.02 | 0.83 | 0.17 | 3.02 |
| 0.05 | 0.83 | 0.17 | 1.03 | 0.83 | 0.17 | 3.03 |
| 0.05 | 0.83 | 0.17 | 1.04 | 0.83 | 0.17 | 3.04 |
| 0.05 | 0.83 | 0.17 | 1.00 | 0.95 | 0.05 | 3.00 |
| 0.05 | 0.83 | 0.17 | 0.97 | 0.95 | 0.05 | 2.97 |
| 0.05 | 0.83 | 0.17 | 0.98 | 0.95 | 0.05 | 2.98 |
| 0.05 | 0.83 | 0.17 | 0.99 | 0.95 | 0.05 | 2.99 |
| 0.05 | 0.83 | 0.17 | 1.01 | 0.95 | 0.05 | 3.01 |
| 0.05 | 0.83 | 0.17 | 1.02 | 0.95 | 0.05 | 3.02 |
| 0.05 | 0.83 | 0.17 | 1.03 | 0.95 | 0.05 | 3.03 |

In the above-mentioned specific examples, “MA” represents methylammonium, “FA” represents formamidinium, “PEA” represents phenethylammonium, “PTA” represents phenyltriethylammonium, “ThMA” represents 2-thiophenemethylammonium, “3BBA” represents 3-bromobenzylammonium, “3AMPY” represents 3-(aminomethyl)pyridine, “PDMA” represents 1,4-phenylenedimethanammonium, “TTDMA” represents thieno[3,2-b] thiophene-2,5-diyldimethanammonium, “4FPEA” represents 4-fluorophenethylammonium, “BA” represents butylammonium, and “TEA” represents 2-thiopheneethylammonium.

The above-mentioned crystal having a perovskite structure preferably has a cubic structure in which the metal atom B, the organic molecules A, and the halogen atom X are arranged on a body-centered position, the respective corners, and a face-centered position, respectively. The details are not clear, but it is assumed that, when such structure is present, the orientation of an octahedron in a crystal lattice can be easily changed, and hence the mobility of an electron in the crystal having a perovskite structure increases, and the photoelectric conversion efficiency of the photoelectric conversion element is improved.

An organic-inorganic perovskite compound to be used in the present disclosure is preferably a crystalline semiconductor. The term “crystalline semiconductor” means a semiconductor that enables the measurement of an X-ray scattering intensity distribution to detect a scattering peak. When the organic-inorganic perovskite compound is the crystalline semiconductor, the mobility of the electron in the organic-inorganic perovskite compound increases, and the photoelectric conversion efficiency of the photoelectric conversion element is improved.

In addition, the photoelectric conversion layer according to the present disclosure may include a material except the crystal having an organic-inorganic perovskite structure to the extent that the photoelectric conversion efficiency and the charge transportability are not impaired.

The thickness of the photoelectric conversion layer according to the present disclosure is preferably 5 to 2,000 nm. When the thickness is 5 nm or more, light can be sufficiently absorbed, and when the thickness is 2,000 nm or less, the generated charge can be transported to the respective electrodes. A more preferred lower limit is 50 nm, a more preferred upper limit is 1,200 nm, a still more preferred lower limit is 100 nm, and a still more preferred upper limit is 1,000 nm.

### [Second Charge-Transporting Layer]

In the present disclosure, the method may include a step of further forming a second charge-transporting layer on the surface of the charge-transporting layer 6 (between the charge-transporting layer 6 and the first electrode 7) from the viewpoint of the compatibility of the film of the charge-transporting layer 6. A method of forming the second charge-transporting layer is as described above.

A material for the second charge-transporting layer is not particularly limited, and examples thereof include a spirofluorene compound, a triphenylamine compound, a chrysene compound, a pyrene compound, a phthalocyanine compound, a carbazole compound, a fluorene compound, a phenylcyclohexane compound, a benzidine compound, a phenoxazine compound, a phenylenediamine compound, a thiocyanate compound, and a thiophene compound. The compound preferably has an aromatic ring from the viewpoint of the compatibility of a film interface, and Spiro-OMeTAD, PTAA, or a phthalocyanine compound is particularly preferred.

In addition, the second charge-transporting layer may contain a dopant as an additive in order to improve its charge-transporting ability. Examples of a substance that may be used as the dopant include lithium compounds such as lithium bis(trifluoromethanesulfonyl)imide, cobalt compounds such as [tris(2-(1H-pyrazol-1-yl)-4-tert-butylpyridine) cobalt(III) tris(bis(trifluoromethylsulfonyl)imide)], boron compounds such as tetrakis(pentafluorophenyl)borate, molybdenum compounds such as tris[1-(methoxycarbonyl)-2-(trifluoromethyl)-ethane-1,2-dithiolene]molybdenum, organic compounds each having a tetracyanoquinodimethane skeleton such as 2,3,4,6-tetrafluoro-7,7,8,8-tetracyanoquinodimethane, and organic compounds each having a pyridine skeleton such as 4-tert-butylpyridine.

### [Electron-Transporting Layer]

In the photoelectric conversion element of the present disclosure, the electron-transporting layer 4 may be arranged between the second electrode 3 and the photoelectric conversion layer 5 as illustrated in FIG. 1.

A material for the electron-transporting layer 4 is not particularly limited, and examples thereof include an N-type conductive polymer, an N-type low-molecular-weight organic semiconductor, an N-type metal oxide, an N-type metal sulfide, a halogenated alkali metal, an alkali metal, and a surfactant. Specific examples thereof include a cyano group-containing polyphenylene vinylene, a boron-containing polymer, bathocuproine, bathophenanthroline, hydroxyquinolinatoaluminum, an oxadiazole compound, a benzimidazole compound, a naphthalenetetracarboxylic acid compound, a fullerene compound, a perylene derivative, a phosphine oxide compound, a phosphine sulfide compound, a fluoro group-containing phthalocyanine, titanium oxide, zinc oxide, indium oxide, tin oxide, gallium oxide, tin sulfide, indium sulfide, and zinc sulfide. In particular, tin oxide may be obtained through the reaction of tin(II) chloride, tin(IV) chloride, tin(II) chloride dihydrate, or tin(IV) chloride pentahydrate.

A preferred lower limit of the thickness of the electron-transporting layer 4 is 1 nm, and a preferred upper limit thereof is 2,000 nm. When such thickness is 1 nm or more, a hole can be sufficiently blocked, and when the thickness is 2,000 nm or less, the electron-transporting layer 4 is less liable to serve as a resistance at the time of the electron transportation, and hence the photoelectric conversion efficiency is improved. A more preferred lower limit of the thickness is 3 nm, a more preferred upper limit thereof is 1,000 nm, a still more preferred lower limit thereof is 5 nm, and a still more preferred upper limit thereof is 500 nm.

### <Photoelectric Conversion Element and Application Examples Thereof>

The photoelectric conversion element produced by the method of producing a photoelectric conversion element of the present disclosure and application examples thereof are described below.

### [Photoelectric Conversion Element]

The photoelectric conversion element of the present disclosure is produced by the above-mentioned method of producing a photoelectric conversion element. The photoelectric conversion element 1 includes, on the substrate 2, the second electrode 3, the electron-transporting layer 4, the photoelectric conversion layer 5, the charge-transporting layer 6, and the first electrode 7. One of the first electrode 7 and the second electrode 3 is an anode, and the other is a cathode. A current can be extracted by connecting the first electrode 7 and the second electrode 3 with an external circuit.

The photoelectric conversion layer 5 is excited by light that has entered the layer through the substrate 2, the second electrode 3, and the electron-transporting layer 4, or the first electrode 7 and the charge-transporting layer 6 to generate an electron or a hole. That is, the photoelectric conversion layer 5 generates a current between the first electrode 7 and the second electrode 3. The electron-transporting layer 4 is a layer arranged between the photoelectric conversion layer 5, and the two electrodes (the second electrode 3 and the first electrode 7), and may be omitted in some cases. A form in which the plurality of electron-transporting layers 4 and photoelectric conversion layers 5 are laminated may be adopted. Such form may also be referred to as “tandem structure.”

The photoelectric conversion element of the present disclosure is characterized by including: the first electrode 7; the second electrode 3; the photoelectric conversion layer 5 arranged between the first electrode 7 and the second electrode 3, the photoelectric conversion layer containing the crystal having a perovskite structure; and the charge-transporting layer 6 between the photoelectric conversion layer 5 and the first electrode 7. In addition, in order to improve the photoelectric conversion efficiency, a tandem type in which the photoelectric conversion elements 1 are laminated may be adopted. The kind of the photoelectric conversion element to be laminated is not limited, and for example, a silicon solar cell or a CIGS solar cell may be adopted in addition to a perovskite solar cell using a crystal having a perovskite structure in the photoelectric conversion layer 5.

In the photoelectric conversion element of the present disclosure, it is preferred that the charge-transporting layer 6 be arranged between the photoelectric conversion layer 5 and the first electrode 7, and the charge-transporting layer 6 contain a charge-transporting particle and a resin, and be arranged on the surface of the photoelectric conversion layer 5. The thickness of the charge-transporting layer 6 is preferably 1 to 1,000 nm, more preferably 5 to 500 nm, particularly preferably 10 to 200 nm.

The thickness of the photoelectric conversion layer is preferably 5 to 2,000 nm. When the thickness is 5 nm or more, light can be sufficiently absorbed, and when the thickness is 2,000 nm or less, the generated charge can be transported to the respective electrodes. A more preferred lower limit is 50 nm, a more preferred upper limit is 1,200 nm, a still more preferred lower limit is 100 nm, and a still more preferred upper limit is 1,000 nm.

In the present disclosure, the presence of each layer may be recognized, for example, by X-ray photoelectron spectroscopy (XPS), nuclear magnetic resonance (NMR), or by energy dispersive X-ray spectroscopy (EDS) using an electron gun of a scanning electron microscope (SEM) or a transmission electron microscope (TEM). The shape and aspect ratio of the charge-transporting particle may be identified with a scanning electron microscope (SEM) or a transmission electron microscope (TEM). In the present disclosure, the thickness of each layer may be identified by, for example, observing a cross section of the photoelectric conversion element with a scanning electron microscope (SEM) or a transmission electron microscope (TEM).

### [Photoelectric Conversion Apparatus]

A photoelectric conversion apparatus of the present disclosure includes the above-mentioned photoelectric conversion element. The photoelectric conversion apparatus may be formed by using the plurality of photoelectric conversion elements of the present disclosure. When the plurality of photoelectric conversion elements are connected, such photoelectric conversion apparatus may also be referred to as “photoelectric conversion cell” or “photoelectric conversion module.”

Photoelectric conversion elements having different absorption wavelengths may be laminated as the photoelectric conversion elements to increase an output voltage. In addition, the photoelectric conversion apparatus includes the photoelectric conversion element of the present disclosure and an inverter. The inverter may be a converter for converting a DC voltage to an AC voltage. The photoelectric conversion apparatus may include an electricity storage unit connected to the photoelectric conversion element. The electricity storage unit is not limited as long as the electricity storage unit can store electricity. Examples thereof include a secondary battery using lithium ions, an all-solid-state battery, and an electric double layer capacitor. In order to impart a function of, for example, maintaining or increasing the amount of incident light, a surface layer to which water or dirt is hard to adhere, or a function of collecting or guiding light may be added.

### [Moving Body]

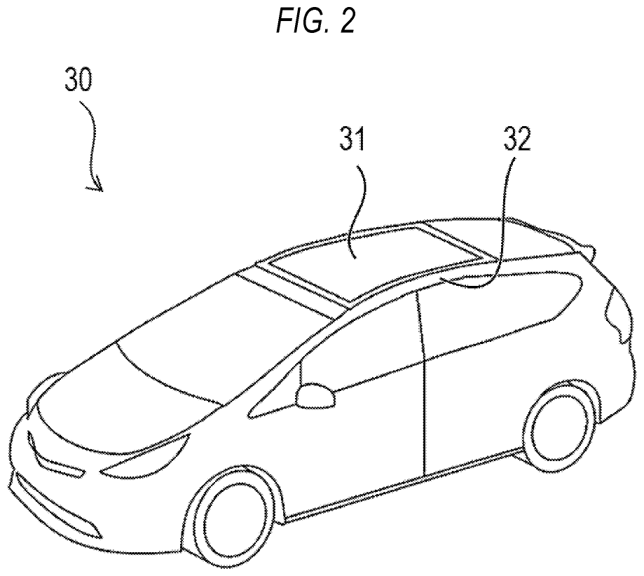

A moving body of the present disclosure includes the above-mentioned photoelectric conversion element. FIG. 2 is a perspective view for schematically illustrating a moving body including the photoelectric conversion element according to one embodiment of the present disclosure. A moving body 30 includes a photoelectric conversion element 31 of the present disclosure and a body 32 including the photoelectric conversion element 31.

The photoelectric conversion element 31 is arranged on the position of the body 32 at which ambient light can be received. When the moving body 30 is an automobile, the photoelectric conversion element 31 may be arranged on a roof. Electric energy obtained by the photoelectric conversion element 31 may serve as the power of the moving body 30 or the power of any other electric equipment. Electric energy generated from the power of the moving body 30 may be used for the power of the photoelectric conversion element 31. When the moving body 30 is an automobile, friction energy generated with a brake may be converted into electric energy to be used for the control of the photoelectric conversion element 31.

The moving body 30 may be, for example, an automobile, a motorcycle, a railway vehicle, a ship, or a flying body including an artificial satellite, an airplane, and a drone. The configuration of the body 32 of the moving body 30 is not particularly limited, but is preferably formed of a material having high strength.

### [Building Material]

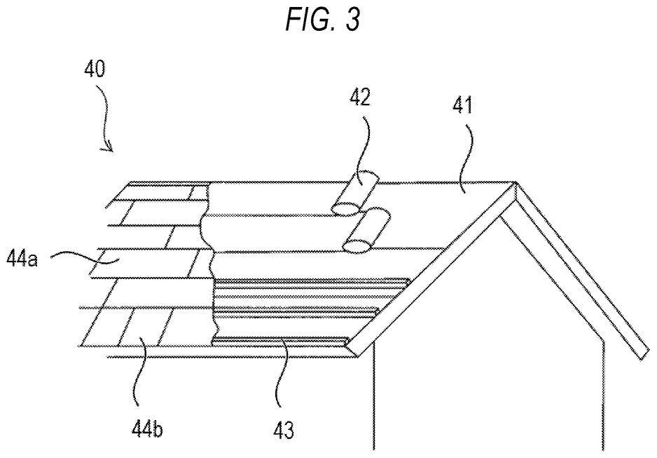

A building material of the present disclosure includes the above-mentioned photoelectric conversion element. FIG. 3 is a perspective view for schematically illustrating a building material including the photoelectric conversion element according to one embodiment of the present disclosure. A building material 40 may be a roof of a building. The building material 40 of this embodiment includes a photoelectric conversion element 41 of the present disclosure, a protective member 42 for protecting the photoelectric conversion element 41, a heat dissipation member 43, and exteriors 44a and 44b.

The building material 40 of the present disclosure may include the heat dissipation member 43 having a thermal conductivity higher than that of the photoelectric conversion element 41. When the building material 40 is used for a roof or the like, the temperature of the photoelectric conversion element 41 may be increased by sunlight, and hence the photoelectric conversion efficiency may be reduced. The reduction of the photoelectric conversion efficiency can be suppressed by using the heat dissipation member 43. Examples of the heat dissipation member 43 include a metal, an alloy, a liquid metal, and a liquid resin.

In addition, the building material 40 of the present disclosure may include the exteriors 44a and 44b. The exterior 44a and the exterior 44b may show different colors, or may show the same color. The exterior 44a and the exterior 44b may be formed of the same member, or may be formed of different members. A paint or a transparent substrate may be used as each of the exteriors. An exterior having small light absorption and a high heat-shielding property is preferred.

In addition to the application examples described above, the following application examples are given: portable devices, such as a calculator, a sensor, and a small solar panel; wearable devices, such as a glasses-type terminal, a watch-type terminal, and a portable medical device; sheet structures supported by a plurality of frames, such as a tent, a plastic greenhouse, and a loading platform of a truck; and structures to be used by being fixed, such as a road surface panel, a floating panel, a building material utilizing the flexibility of a substrate, a wall-type building material, a glass-type building material, and a mega solar panel.

### EXAMPLES

The present disclosure is described in more detail below by way of Examples and Comparative Examples. The present disclosure is by no means limited to the following Examples without departing from the gist thereof. In the description of the following Examples, the term “part(s)” is by mass unless otherwise specified.

### <Production of Particle 1>

### Step (1)

Under a nitrogen flow atmosphere, 5.46 parts of orthophthalonitrile and 45 parts of α-chloronaphthalene were loaded into a reaction kettle. After that, the mixture was heated so that its temperature was increased to 30° C., followed by the maintenance of the temperature. Next, 3.75 parts of gallium trichloride was loaded into the mixture at the temperature (30° C.). The moisture concentration of the mixed liquid at the time of the loading was 150 ppm. After that, the temperature of the mixed liquid was increased to 200° C.

Next, under a nitrogen flow atmosphere, the mixed liquid was subjected to a reaction at a temperature of 200° C. for 4.5 hours, and was then cooled. The product was filtered when its temperature reached 150° C. The resultant filter residue was subjected to dispersion washing with N,N-dimethylformamide at a temperature of 140° C. for 2 hours, and was then filtered. The resultant filter residue was washed with methanol, and was then dried to provide a chlorogallium phthalocyanine particle in a yield of 71 mass %.

### Step (2)

4.65 Parts of the chlorogallium phthalocyanine particle was dissolved in 139.5 parts of concentrated sulfuric acid at a temperature of 10° C., and the solution was dropped into 620 parts of ice water under stirring so that the particle was reprecipitated, followed by filtration with a filter press under reduced pressure. At this time, No. 5C (manufactured by Advantec Toyo Kaisha, Ltd.) was used as a filter. The resultant wet cake (filter residue) was subjected to dispersion washing with 2% ammonia water for 30 minutes, and was then filtered with the filter press.

Next, the resultant wet cake (filter residue) was subjected to dispersion washing with ion-exchanged water, and then its filtration with the filter press was repeated three times. Finally, the filter residue was freeze-dried to provide a hydroxygallium phthalocyanine particle (hydrous hydroxygallium phthalocyanine particle) having a solid content of 23 mass % in a yield of 71 mass %. The hydroxygallium phthalocyanine particle was dried with a hyper-dry dryer (product name: HD-06R, frequency (oscillatory frequency): 2,455 MHz±15 MHz, manufactured by Biocon (Japan) Ltd.). Thus, a hydroxygallium phthalocyanine (OHGaPc) particle (crystal) having a water content of 1.0 mass % or less was obtained.

### Step (3)

5 Parts of the hydroxygallium phthalocyanine particle was mixed with 5 parts of an N-methylformamide solvent, and the mixture was subjected to dispersion treatment for 6 hours with a sand mill (TSG-1/4G-4U, manufactured by Igarashi Machine Production Co., Ltd. (currently AIMEX Co., Ltd.), disc diameter: 70 mm, number of discs: 5) having sealed therein 5 parts of glass beads, followed by filtration and drying to provide a particle 1 (specific gravity: 1.6). The aspect ratio of the particle was 3 or less.

### <Production of Resin Solution 1>

1.0 Gram of a polyvinyl acetal resin (product name: S-LEC (trademark) BM-2, manufactured by Sekisui Chemical Co., Ltd., glass transition temperature: 71° C.) was dissolved in 19 g of 2-propanol serving as a solvent under stirring for 24 hours to provide a resin solution 1.

### <Production of Resin Solution 2>

1.0 Gram of a polyvinyl acetal resin (product name: S-LEC (trademark) BX-1, manufactured by Sekisui Chemical Co., Ltd., glass transition temperature: 95° C.) was dissolved in 19 g of ethanol serving as a solvent under stirring for 24 hours to provide a resin solution 2.

### <Production of Resin Solution 3>

1.0 Gram of a polyvinyl acetal resin (product name: S-LEC (trademark) BL-S, manufactured by Sekisui Chemical Co., Ltd., glass transition temperature: 66° C.) was dissolved in 19 g of 2-propanol serving as a solvent under stirring for 24 hours to provide a resin solution 3.

### <Production of Resin Solution 4>

1.0 Gram of poly(methyl methacrylate) (PMMA, manufactured by Sigma-Aldrich Co. LLC, glass transition temperature: 70° C.) was dissolved in 19 g of chlorobenzene serving as a solvent under stirring for 24 hours to provide a resin solution 4.

### <Production of Resin Solution 5>

1.0 Gram of poly(methyl methacrylate) (PMMA, manufactured by Sigma-Aldrich Co. LLC, glass transition temperature: 100° C.) was dissolved in 19 g of chlorobenzene serving as a solvent under stirring for 24 hours to provide a resin solution 5.

### <Production of Resin Solution 6>

1.0 Gram of poly(3-hexylthiophene-2,5-diyl) (P3HT, glass transition temperature: 130° C.) was dissolved in 19 g of chlorobenzene serving as a solvent under stirring for 24 hours to provide a resin solution 6.

### <Production of Resin Solution 7>

1.0 Gram of poly(4-vinylpyridine) (glass transition temperature: 137° C.) was dissolved in 19 g of 2-propanol serving as a solvent under stirring for 24 hours to provide a resin solution 7.

### <Production of Resin Solution 8>

1.0 Gram of poly[9,9-bis-(2-ethylhexyl)-9H-fluorene-2,7-diyl] (glass transition temperature: 45° C.) was dissolved in 19 g of chlorobenzene serving as a solvent under stirring for 24 hours to provide a resin solution 8.

### <Production of Resin Solution 9>

1.0 Gram of poly(3-hexylthiophene-2,5-diyl) (P3HT, glass transition temperature: 130° C.) was dissolved in 19 g of dichlorobenzene serving as a solvent under stirring for 24 hours to provide a resin solution 9.

### <Production of Resin Solution 10>

1.0 Gram of a polyvinyl acetal resin (product name: S-LEC (trademark) BL-S, manufactured by Sekisui Chemical Co., Ltd., glass transition temperature: 66° C.) was dissolved in 19 g of acetone serving as a solvent under stirring for 24 hours to provide a resin solution 10.

### Example 1

### [Formation of Electron-Transporting Layer]

A glass substrate with ITO was washed, and a tin(II) oxide colloidal solution (15% water dispersion, manufactured by Alfa Aesar) diluted fivefold was applied thereonto by spin coating, followed by heating at 150° C. for 30 minutes to form an electron-transporting layer as a thin film having a thickness of 16 nm.

### [Formation of Photoelectric Conversion Layer]

1.1 Grams of lead iodide and 0.39 g of methylammonium iodide were dissolved in 0.36 g of N,N-dimethylformamide and 1.45 g of dimethyl sulfoxide to prepare a photoelectric conversion layer coating liquid. The coating liquid was applied onto the electron-transporting layer by spin coating to form a photoelectric conversion layer formed of MAPbI₃ and having a thickness of 500 nm.

### [Formation of Charge-Transporting Layer]

0.1 Gram of the particle 1 and 0.01 g of a calixarene compound (Japanese Patent Laid-Open No. 2003-207913) were mixed with 10.6 g of 2-propanol serving as a solvent, and 11 g of beads (zirconia beads, TORAYCERAM (trademark) zirconia beads, 0.3 mm) were loaded into the mixture, followed by the dispersion of the materials in the solvent with a paint shaker (manufactured by Toyo Seiki Seisaku-sho, Ltd.) for 3 hours. After that, 0.2 g of the resin solution 1 was added to the dispersion liquid, and dispersion was performed with the paint shaker again for 4 hours to prepare a charge-transporting layer coating liquid. The charge-transporting layer coating liquid was applied onto the photoelectric conversion layer by spin coating to form a charge-transporting layer having a thickness of 180 nm.

### [Introduction of Second Charge-Transporting Layer]

0.15 Gram of Spiro-OMeTAD serving as a material for a second charge-transporting layer was dissolved in 2.2 g of chlorobenzene. 36 Microliters of an acetonitrile solution obtained by dissolving 0.2 g of lithium bis(trifluoromethanesulfonyl)imide in 0.3 g of acetonitrile and 60 μL of 4-tert-butylpyridine (TBP) were added to the chlorobenzene solution, and the contents were mixed. Further, 58 μL of an acetonitrile solution obtained by dissolving 0.11 g of [tris(2-(1H-pyrazol-1-yl)-4-tert-butylpyridine) cobalt(III) tris(bis(trifluoromethylsulfonyl)imide)] in 0.3 g of acetonitrile was mixed thereinto to prepare a material solution for a second charge-transporting layer. The material solution was applied onto the above-mentioned charge-transporting layer by a spin coating method to form a second charge-transporting layer having a thickness of 150 nm.

### [Formation of First Electrode]

A gold electrode having a thickness of 80 nm and an area of 0.36 cm² was formed on the second charge-transporting layer by a vacuum vapor deposition method. Thus, a photoelectric conversion element was obtained.

### [Analysis of Compound Amount]

The electrode surface of the photoelectric conversion element was peeled off so that the surface of the charge-transporting layer thereof was exposed. The surface of the charge-transporting layer was wiped with a cotton swab or the like with a solvent, dissolved in deuterated sulfuric acid, and subjected to ¹H-NMR measurement (apparatus: AVANCE III 500 manufactured by BRUKER). In addition, the presence of a compound was determined by subjecting the peeled-off charge-transporting layer components to mass and structure analyses through GPC, MALDI-TOF-MS, IR, gas chromatography, or elemental analysis, such as XPS or EDX.

In addition, the thickness of the layer was determined with a sectional SEM (apparatus: SmartSEM manufactured by Carl Zeiss Co., Ltd.) after the cutting of the photoelectric conversion element and the fixing of the sample to a tilted sample stage.

### [Analysis of Particle Diameter of Charge-Transporting Particle]

The particle diameter of the charge-transporting particle in the charge-transporting layer coating liquid was determined by measuring the Z-average particle diameter thereof with the above-mentioned Zetasizer Nano ZS (manufactured by Malvern Panalytical Ltd.).

### Example 2

A photoelectric conversion element is obtained in the same manner as in Example 1 except that the addition amount of the resin solution 1 is changed to 0.25 g in the preparation of the charge-transporting layer coating liquid.

### Example 3

A photoelectric conversion element is obtained in the same manner as in Example 1 except that the addition amount of the resin solution 1 is changed to 0.40 g in the preparation of the charge-transporting layer coating liquid.

### Example 4

A photoelectric conversion element is obtained in the same manner as in Example 1 except that the addition amount of the resin solution 1 is changed to 0.10 g.

### Example 5

A photoelectric conversion element is obtained in the same manner as in Example 1 except that the addition amount of the resin solution 1 is changed to 0.50 g in the preparation of the charge-transporting layer coating liquid.

### Example 6

A photoelectric conversion element is obtained in the same manner as in Example 1 except that the addition amount of the resin solution 1 is changed to 0.08 g in the preparation of the charge-transporting layer coating liquid.

### Example 7

A photoelectric conversion element is obtained in the same manner as in Example 1 except the following the time of the paint shaker dispersion before the addition of the resin solution 1 is changed to 6 hours, the particle diameter in the resultant dispersion liquid is reduced by centrifuging the dispersion liquid (15,000 rpm, 2 minutes), then the resin solution 1 is added so that the mass ratio of the charge-transporting particle to the resin may become 10 times, and the paint shaker dispersion is carried out again for 4 hours, in the preparation of the charge-transporting layer coating liquid.

### Example 8

A photoelectric conversion element is obtained in the same manner as in Example 1 except that the time of the paint shaker dispersion before the addition of the resin solution 1 is changed to 1 hour and the time of the paint shaker dispersion after the addition of the resin solution 1 is changed to 1 hour, in the preparation of the charge-transporting layer coating liquid.

### Example 9

A photoelectric conversion element is obtained in the same manner as in Example 1 except that 10.6 g of 2-propanol is changed to 10.6 g of ethanol and the resin solution 1 is changed to the resin solution 2, in the preparation of the charge-transporting layer coating liquid.

### Example 10

A photoelectric conversion element is obtained in the same manner as in Example 1 except that the particle 1 is changed to a copper phthalocyanine particle in the preparation of the charge-transporting layer coating liquid.

### Example 11

A photoelectric conversion element is obtained in the same manner as in Example 1 except that the particle 1 is changed to a tetraphenylporphyrin (TPP) particle in the preparation of the charge-transporting layer coating liquid.

### Example 12

A photoelectric conversion element is obtained in the same manner as in Example 1 except that the particle 1 is changed to a quinacridone particle in the preparation of the charge-transporting layer coating liquid.

### Example 13

A photoelectric conversion element is obtained in the same manner as in Example 1 except that the particle 1 is changed to a particle containing a compound represented by the following formula (Pc-3) in the preparation of the charge-transporting layer coating liquid.

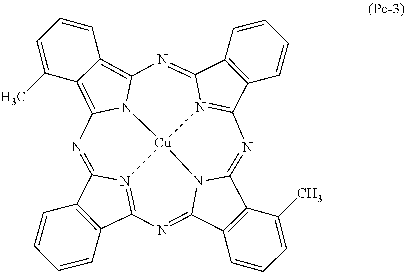

### Example 14

A photoelectric conversion element is obtained in the same manner as in Example 1 except that the resin solution 1 is changed to the resin solution 4 in the preparation of the charge-transporting layer coating liquid.

### Example 15

A photoelectric conversion element is obtained in the same manner as in Example 1 except that the resin solution 1 is changed to the resin solution 5 in the preparation of the charge-transporting layer coating liquid.

### Example 16

A photoelectric conversion element is obtained in the same manner as in Example 1 except that the resin solution 1 is changed to the resin solution 3 in the preparation of the charge-transporting layer coating liquid.

### Example 17

A photoelectric conversion element is obtained in the same manner as in Example 1 except that the resin solution 1 is changed to the resin solution 6 in the preparation of the charge-transporting layer coating liquid.

### Example 18

A photoelectric conversion element is obtained in the same manner as in Example 1 except that the resin solution 1 is changed to the resin solution 7 in the preparation of the charge-transporting layer coating liquid.

### Example 19

A photoelectric conversion element is obtained in the same manner as in Example 1 except that the addition amount of the resin solution 1 is set to 0.25 g and the second charge-transporting layer is not arranged, in the preparation of the charge-transporting layer coating liquid.

### Example 20

A photoelectric conversion element is obtained in the same manner as in Example 1 except that the solvent is changed to 4.2 g of 2-propanol and 6.4 g of chlorobenzene, in the preparation of the charge-transporting layer coating liquid.

### Example 21

A photoelectric conversion element is obtained in the same manner as in Example 1 except that 10.6 g of 2-propanol is changed to 10.6 g of methanol in the preparation of the charge-transporting layer coating liquid.

### Example 22

A photoelectric conversion element is obtained in the same manner as in Example 1 except that 10.6 g of 2-propanol is changed to 10.6 g of 1-butanol in the preparation of the charge-transporting layer coating liquid.

### Example 23

A photoelectric conversion element is obtained in the same manner as in Example 1 except that 10.6 g of 2-propanol is changed to 10.6 g of 1-pentanol in the preparation of the charge-transporting layer coating liquid.

### Example 24

A photoelectric conversion element is obtained in the same manner as in Example 1 except that 10.6 g of 2-propanol is changed to 10.6 g of benzyl alcohol in the preparation of the charge-transporting layer coating liquid.

### Example 25

A photoelectric conversion element is obtained in the same manner as in Example 1 except that the resin solution 1 is changed to the resin solution 8 and its addition amount is changed to 0.14 g, in the preparation of the charge-transporting layer coating liquid.

### Example 26

A photoelectric conversion element is obtained in the same manner as in Example 1 except that the photoelectric conversion layer coating liquid is changed to the following to form a photoelectric conversion layer.

0.487 Gram of methylammonium bromide, 1.034 g of formamidinium iodide, 2.903 g of lead iodide, and 0.139 g of methylammonium bromide are dissolved in 4.25 g of N,N-dimethylformamide and 1.216 g of dimethyl sulfoxide, and are stirred for 1 hour (solution 1). In addition, 0.100 g of cesium iodide is dissolved in 0.285 g of dimethyl sulfoxide, and the solution is stirred for 1 hour (solution 2). After that, the dissolved cesium iodide solution (solution 2) is added to the solution 1 to prepare a photoelectric conversion layer coating liquid. The coating liquid is applied onto the electron-transporting layer by spin coating to form a photoelectric conversion layer formed of Cs_{0.05} (FA_{0.83}MA_{0.17})_{0.95}Pb(I_{0.83}Br_{0.17})₃ and having a thickness of 600 nm.

### Comparative Example 1

A photoelectric conversion element was obtained in the same manner as in Example 1 except that the resin solution 1 was not used in the preparation of the charge-transporting layer coating liquid.

### Comparative Example 2

A photoelectric conversion element is obtained in the same manner as in Example 1 except that the particle 1 is changed to a particle containing a compound represented by the following formula (Pc-4), 10.6 g of 2-propanol is changed to 10.6 g of dichlorobenzene, the resin solution 1 is changed to the resin solution 9, and the solvent is set to only dichlorobenzene, in the preparation of the charge-transporting layer coating liquid.

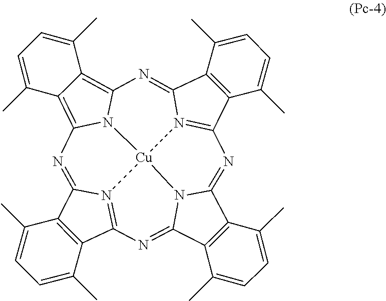

### Comparative Example 3

A photoelectric conversion element is obtained in the same manner as in Example 1 except following 10.6 g of 2-propanol is changed to 10.6 g of acetone, the resin solution 1 is changed to the resin solution 10, and the solvent is set to only acetone, in the preparation of the charge-transporting layer coating liquid.

### Comparative Example 4

A photoelectric conversion element is obtained in the same manner as in Example 1 except that 10.6 g of 2-propanol is changed to 10.6 g of chlorobenzene (the solvent is set to only chlorobenzene) and the resin solution 1 is not used, in the preparation of the charge-transporting layer coating liquid.

### Comparative Example 5

A photoelectric conversion element is obtained in the same manner as in Example 1 except that, in the preparation of the charge-transporting layer coating liquid, the particle 1 is changed to a nickel (II) phthalocyanine-tetrasulfonic acid tetrasodium salt, 10.6 g of 2-propanol is changed to 10.6 g of water, the resin solution 1 is changed to PEDOT:PSS (2.7 mass %, manufactured by Sigma-Aldrich Co. LLC), and its addition amount is changed to 0.37 g, and the solvent is set to only water.

### Comparative Example 6

A photoelectric conversion element is obtained in the same manner as in Example 1 except that, in the preparation of the charge-transporting layer coating liquid, 10.6 g of 2-propanol is changed to 10.6 g of chlorobenzene, the particle 1 is changed to Spiro-OMeTAD, the resin solution 1 is changed to the resin solution 5, and the solvent is set to only chlorobenzene.

### [Evaluation]

A power source (manufactured by Keithley Instruments, Model 236) was connected between the electrodes of the photoelectric conversion element produced in Example 1, and constant light was applied thereto with a solar simulator (manufactured by Yamashita Denso Corporation) at an intensity of 100 mW/cm², followed by the measurement of a current and a voltage to be generated. Thus, the photoelectric conversion efficiency of the element was measured. The result is shown in Table 2.

Examples 2 to 26 and Comparative Examples 1 to 6 are each also evaluated for its photoelectric conversion efficiency in the same manner as in Example 1. The results are shown in Table 2.

In Table 2, the photoelectric conversion efficiency in Example 1 is set to 100, and a ratio thereto is shown as the conversion efficiency of each photoelectric conversion element.

In each of Comparative Examples 5 and 6, the charge-transporting material is dissolved in the solvent and the particle is not in a dispersed state, and hence the particle diameter is not measured.

TABLE 2

| | | | | Resin | | | Mass ratio of | |
|---|---|---|---|---|---|---|---|---|
| | Charge-transporting material | | | | Glass | | charge- | Photoelectric |
| | | | Particle | | transition | | transporting | conversion |
| | | Particle | diameter | | temperature | Solvent | material to | efficiency |
| | Compound | or not | [nm] | Compound | [° C.] | Compound | resin | [%] |
| Example 1 | Hydroxygallium | Particle | 2.0 × 10² | BM-2 | 71° C. | 2-Propanol | 10 | 100.0 |
| | phthalocyanine | | | | | | | |
| Example 2 | Hydroxygallium | Particle | 1.9 × 10² | BM-2 | 71° C. | 2-Propanol | 8 | 98.9 |
| | phthalocyanine | | | | | | | |
| Example 3 | Hydroxygallium | Particle | 1.9 × 10² | BM-2 | 71° C. | 2-Propanol | 5 | 97.3 |
| | phthalocyanine | | | | | | | |
| Example 4 | Hydroxygallium | Particle | 2.1 × 10² | BM-2 | 71° C. | 2-Propanol | 20 | 98.4 |
| | phthalocyanine | | | | | | | |
| Example 5 | Hydroxygallium | Particle | 1.9 × 10² | BM-2 | 71° C. | 2-Propanol | 4 | 89.7 |
| | phthalocyanine | | | | | | | |
| Example 6 | Hydroxygallium | Particle | 2.0 × 10² | BM-2 | 71° C. | 2-Propanol | 25 | 90.8 |
| | phthalocyanine | | | | | | | |
| Example 7 | Hydroxygallium | Particle | 1.5 × 10¹ | BM-2 | 71° C. | 2-Propanol | 10 | 97.8 |
| | phthalocyanine | | | | | | | |
| Example 8 | Hydroxygallium | Particle | 4.8 × 10² | BM-2 | 71° C. | 2-Propanol | 10 | 98.4 |
| | phthalocyanine | | | | | | | |
| Example 9 | Hydroxygallium | Particle | 2.5 × 10² | BX-1 | 95° C. | Ethanol | 10 | 97.8 |
| | phthalocyanine | | | | | | | |
| Example 10 | Copper | Particle | 4.2 × 10² | BM-2 | 71° C. | 2-Propanol | 10 | 94.1 |
| | phthalocyanine | | | | | | | |
| Example 11 | Tetraphenylporphyrin | Particle | 3.3 × 10² | BM-2 | 71° C. | 2-Propanol | 10 | 88.1 |
| Example 12 | Quinacridone | Particle | 3.0 × 10² | BM-2 | 71° C. | 2-Propanol | 10 | 87.0 |
| Example 13 | (Pc-3) | Particle | 2.6 × 10² | BM-2 | 71° C. | 2-Propanol | 10 | 89.7 |
| Example 14 | Hydroxygallium | Particle | 2.2 × 10² | PMMA | 70° C. | 2-Propanol/ | 10 | 91.4 |
| | phthalocyanine | | | | | chlorobenzene | | |
| Example 15 | Hydroxygallium | Particle | 2.3 × 10² | PMMA | 100° C. | 2-Propanol/ | 10 | 87.0 |
| | phthalocyanine | | | | | chlorobenzene | | |
| Example 16 | Hydroxygallium | Particle | 2.0 × 10² | BL-S | 66° C. | 2-Propanol | 10 | 97.8 |
| | phthalocyanine | | | | | | | |
| Example 17 | Hydroxygallium | Particle | 2.9 × 10² | P3HT | 130° C. | 2-Propanol/ | 10 | 87.0 |
| | phthalocyanine | | | | | chlorobenzene | | |
| Example 18 | Hydroxygallium | Particle | 2.2 × 10² | Poly(4- | 137° C. | 2-Propanol | 10 | 86.5 |
| | phthalocyanine | | | vinylpyridine) | | | | |
| Example 19 | Hydroxygallium | Particle | 1.8 × 10² | BM-2 | 71° C. | 2-Propanol | 8 | 85.9 |
| | phthalocyanine | | | | | | | |
| Example 20 | Hydroxygallium | Particle | 2.9 × 10² | BM-2 | 71° C. | 2-Propanol/ | 10 | 92.4 |
| | phthalocyanine | | | | | chlorobenzene | | |
| Example 21 | Hydroxygallium | Particle | 1.7 × 10² | BM-2 | 71° C. | 2-Propanol/methanol | 10 | 97.8 |
| | phthalocyanine | | | | | | | |
| Example 22 | Hydroxygallium | Particle | 2.1 × 10² | BM-2 | 71° C. | 2-Propanol/1-butanol | 10 | 96.8 |
| | phthalocyanine | | | | | | | |
| Example 23 | Hydroxygallium | Particle | 2.2 × 10² | BM-2 | 71° C. | 2-Propanol/1-pentanol | 10 | 96.2 |
| | phthalocyanine | | | | | | | |
| Example 24 | Hydroxygallium | Particle | 3.0 × 10² | BM-2 | 71° C. | 2-Propanol/benzyl | 10 | 91.9 |
| | phthalocyanine | | | | | alcohol | | |
| Example 25 | Hydroxygallium | Particle | 2.9 × 10² | Poly[9,9-bis- | 45° C. | 2-Propanol/ | 14 | 83.8 |
| | phthalocyanine | | | (2-ethylhexyl)- | | chlorobenzene | | |
| | | | | 9H-fluorene- | | | | |
| | | | | 2,7-diyl] | | | | |
| Example 26 | Hydroxygallium | Particle | 2.0 × 10² | BM-2 | 71° C. | 2-Propanol | 10 | 99.5 |
| | phthalocyanine | | | | | | | |
| Comparative | Hydroxygallium | Particle | 1.9 × 10² | None | — | 2-Propanol | — | 70.3 |
| Example 1 | phthalocyanine | | | | | | | |
| Comparative | (Pc-4) | Particle | 3.8 × 10² | P3HT | 130° C. | Dichlorobenzene | 10 | 72.4 |
| Example 2 | | | | | | | | |
| Comparative | Hydroxygallium | Particle | 7.4 × 10² | BL-S | 66° C. | Acetone | 10 | 59.5 |
| Example 3 | phthalocyanine | | | | | | | |
| Comparative | Hydroxygallium | Particle | 4.6 × 10² | None | — | Chlorobenzene | — | 67.6 |
| Example 4 | phthalocyanine | | | | | | | |
| Comparative | Nickel(II) | Not | Not | PEDOT:PSS | — | Water | 10 | 0.0 |
| Example 5 | phthalocyanine- | particle | measured | | | | | |
| | tetrasulfonic acid | | | | | | | |
| | tetrasodium salt | | | | | | | |
| Comparative | Spiro-OMeTAD | Not | Not | PMMA | 100° C. | Chlorobenzene | 10 | 64.9 |
| Example 6 | | particle | measured | | | | | |

According to the present disclosure, the method of producing a photoelectric conversion element improved in photoelectric conversion efficiency can be provided.

While the present disclosure has been described with reference to embodiments, it is to be understood that the present disclosure is not limited to the disclosed embodiments. The scope of the following claims is to be accorded the broadest interpretation so as to encompass all such modifications and equivalent structures and functions.

1. A method of producing a photoelectric conversion element comprising a first electrode, a photoelectric conversion layer containing a crystal having a perovskite structure, a charge-transporting layer, and a second electrode, the method comprising:
applying a charge-transporting layer coating liquid onto a surface of the photoelectric conversion layer to form the charge-transporting layer,
wherein the charge-transporting layer coating liquid contains an alcohol liquid, a resin, and a charge-transporting particle, and
wherein a mass of the charge-transporting particle in the charge-transporting layer coating liquid is 5 to 20 times with respect to a mass of the resin in the charge-transporting layer coating liquid.

2. The method of producing a photoelectric conversion element according to claim 1, wherein the alcohol liquid contains an aliphatic alcohol.

3. The method of producing a photoelectric conversion element according to claim 1, wherein the alcohol liquid contains an alcohol having 5 or less carbon atoms.

4. The method of producing a photoelectric conversion element according to claim 1, wherein the resin is a resin having a Lewis basic functional group.

5. The method of producing a photoelectric conversion element according to claim 1, wherein the resin has at least one functional group selected from the group consisting of a hydroxy group, a carbonyl group, an ether group, an ester group, a pyridyl group and a thienylidene group.

6. The method of producing a photoelectric conversion element according to of claim 1, wherein the resin contains a polyvinyl acetal resin.

7. The method of producing a photoelectric conversion element according to claim 1, wherein the resin has a glass transition temperature of 95° C. or less.

8. The method of producing a photoelectric conversion element according to claim 1, wherein the charge-transporting particle in the charge-transporting layer coating liquid has a particle diameter of 1.0×10¹ to 5.0×10² nm.

9. The method of producing a photoelectric conversion element according to claim 1, wherein the charge-transporting particle is a particle containing a cyclic conjugated compound in which a plurality of pyrrole rings are bonded by conjugated bonds.

10. The method of producing a photoelectric conversion element according to claim 1, wherein the charge-transporting particle is a particle containing a phthalocyanine compound.

11. The method of producing a photoelectric conversion element according to claim 1, wherein the charge-transporting particle is a particle having a structure represented by the following formula (Pc-2): 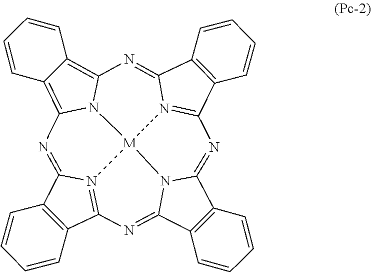
in the formula (Pc-2), M represents H₂, a metal atom having a ligand, or a metal atom free of a ligand.

12. The method of producing a photoelectric conversion element according to claim 1, comprising forming a second charge-transporting layer on a surface of the charge-transporting layer.
